SEQUENCE LISTING (8 sequences)

SEQ ID NO: 1 - DNA, 5121 bp, Artificial Sequence
  Description of Artificial Sequence; note = synthetic construct
gcgaggaggaaacggtgccggagcgcgcagggcttgctgccgccaccgccgctgcacaggctgccggagcgagcctgccgcgcgccgccctccccgctctccttcctgggcgagctgcggggatggggcggccgcgggagcccgagcgcgcgcaggaaccgccgccgccgccgcccgcgtctccgttgccgcgcgcctgagccgccgtcgccgccgcgcgccctgcccgggggcggcccccccagccccatggaggtctcccggaggaaggcgccgccgcgccccccgcgccccgcagcgccactgcccctgctcgcctatctgctggcactggcggctcccggccggggcgcggacgagcccgtgtggcggtcggagcaagccatcggagccatcgcggcgagccaggaggacggcgtgtttgtggcgagcggcagctgcctggaccagctggactacagcctggagcacagcctctcgcgcctgtaccgggaccaagcgggcaactgcacagagccggtctcgctggcgccccccgcgcggccccggcccgggagcagcttcagcaagctgctgctgccctaccgcgagggggcggccggcctcggggggctgctgctcaccggctggaccttcgaccggggcgcctgcgaggtgcggcccctgggcaacctgagccgcaactccctgcgcaacggcaccgaggtggtgtcgtgccacccgcagggctcgacggccggcgtggtgtaccgcgcgggccggaacaaccgctggtacctggcggtggccgccacctacgtgctgcctgagccggagacggcgagccgctgcaaccccgcggcatccgaccacgacacggccatcgcgctcaaggacacggaggggcgcagcctggccacgcaggagctggggcgcctcaagctgtgcgagggcgcgggcagcctgcacttcgtggacgcctttctctggaacggcagcatctacttcccctactacccctacaactatacgagcggcgctgccaccggctggcccagcatggcgcgcatcgcgcagagcaccgaggtgctgttccagggccaggcatccctcgactgcggccacggccaccccgacggccgccgcctgctcctctcctccagcctagtggaggccctggacgtctgggcgggagtgttcagcgcggccgctggagagggccaggagcggcgctcccccaccaccacggcgctctgcctcttcagaatgagtgagatccaggcgcgcgccaagagggtcagctgggacttcaagacggccgagagccactgcaaagaaggggatcaacctgaaagagtccaaccaatcgcatcatctaccttgatccattccgacctgacatccgtttatggcaccgtggtaatgaacaggactgttttattcttggggactggagatggccagttacttaaggttattcttggtgagaatttgacttcaaattgtccagaggttatctatgaaattaaagaagagacacctgttttctacaaactcgttcctgatcctgtgaagaatatctacatttatctaacagctgggaaagaggtgaggagaattcgtgttgcaaactgcaataaacataaatcctgttcggagtgtttaacagccacagaccctcactgcggttggtgccattcgctacaaaggtgcacttttcaaggagattgtgtacattcagagaacttagaaaactggctggatatttcgtctggagcaaaaaagtgccctaaaattcagataattcgaagcagtaaagaaaagactacagtgactatggtgggaagcttctctccaagacactcaaagtgcatggtgaagaatgtggactctagcagggagctctgccagaataaaagtcagcccaaccggacctgcacctgtagcatcccaaccagagcaacctacaaagatgtttcagttgtcaacgtgatgttctccttcggttcttggaatttatcagacagattcaactttaccaactgctcatcattaaaagaatgcccagcatgcgtagaaactggctgcgcgtggtgtaaaagtgcaagaaggtgtatccaccccttcacagcttgcgacccttctgattatgagagaaaccaggaacagtgtccagtggctgtcgagaagacatcaggaggaggaagacccaaggagaacaaggggaacagaaccaaccaggctttacaggtcttctacattaagtccattgagccacagaaagtatcgacattagggaaaagcaacgtgatagtaacgggagcaaactttacccgggcatcgaacatcacaatgatcctgaaaggaaccagtacctgtgataaggatgtgatacaggttagccatgtgctaaatgacacccacatgaaattctctcttccatcaagccggaaagaaatgaaggatgtgtgtatccagtttgatggtgggaactgctcttctgtgggatccttatcctacattgctctgccacattgttcccttatatttcctgctaccacctggatcagtggtggtcaaaatataaccatgatgggcagaaattttgatgtaattgacaacttaatcatttcacatgaattaaaaggaaacataaatgtctctgaatattgtgtggcgacttactgcgggtttttagcccccagtttaaagagttcaaaagtgcgcacgaatgtcactgtgaagctgagagtacaagacacctacttggattgtggaaccctgcagtatcgggaggaccccagattcacggggtatcgggtggaatccgaggtggacacagaactggaagtgaaaattcaaaaagaaaatgacaacttcaatatttccaaaaaagacattgaaattactctcttccatggggaaaatgggcaattaaattgcagttttgaaaatattactagaaatcaagatcttaccaccatcctttgcaaaattaaaggcatcaagactgcaagcaccattgccaactcttctaagaaagttcgggtcaagctgggaaacctggagctctacgtcgagcaggagtcagttccttccacatggtattttctgattgtgctccctgtcttgctagtgattgtcatttttgcggccgtgggggtgaccaggcacaaatcgaaggagctgagtcgcaaacagagtcaacaactagaattgctggaaagcgagctccggaaagagatacgtgacggctttgctgagctgcagatggataaattggatgtggttgatagttttggaactgttcccttccttgactacaaacattttgctctgagaactttcttccctgagtcaggtggcttcacccacatcttcactgaagatatgcataacagagacgccaacgacaagaatgaaagtctcacagctttggatgccctaatctgtaataaaagctttcttgttactgtcatccacacccttgaaaagcagaagaacttttctgtgaaggacaggtgtctgtttgcctccttcctaaccattgcactgcaaaccaagctggtctacctgaccagcatcctagaggtgctgaccagggacttgatggaacagtgtagtaacatgcagccgaaactcatgctgagacgcacggagtccgtcgtcgaaaaactcctcacaaactggatgtccgtctgcctttctggatttctccgggagactgtcggagagcccttctatttgctggtgacgactctgaaccagaaaattaacaagggtcccgtggatgtaatcacttgcaaagccctgtacacacttaatgaagactggctgttgtggcaggttccggaattcagtactgtggcattaaacgtcgtctttgaaaaaatcccggaaaacgagagtgcagatgtctgtcggaatatttcagtcaatgttctcgactgtgacaccattggccaagccaaagaaaagattttccaagcattcttaagcaaaaatggctctccttatggacttcagcttaatgaaattggtcttgagcttcaaatgggcacacgacagaaagaacttctggacatcgacagttcctccgtgattcttgaagatggaatcaccaagctaaacaccattggccactatgagatatcaaatggatccactataaaagtctttaagaagatagcaaattttacttcagatgtggagtactcggatgaccactgccatttgattttaccagattcggaagcattccaagatgtgcaaggaaagagacatcgagggaagcacaagttcaaagtaaaagaaatgtatctgacaaagctgctgtcgaccaaggtggcaattcattctgtgcttgaaaaactttttagaagcatttggagtttacccaacagcagagctccatttgctataaaatacttttttgactttttggacgcccaggctgaaaacaaaaaaatcacagatcctgacgtcgtacatatttggaaaacaaacagccttcctcttcgcttctgggtaaacatcctgaagaaccctcagtttgtctttgacattaagaagacaccacatatagacggctgtttgtcagtgattgcccaggcattcatggatgcattttctctcacagagcagcaactagggaaggaagcaccaactaataagcttctctatgccaaggatatcccaacctacaaagaagaagtaaaatcttattacaaagcaatcagggatttgcctccattgtcatcctcagaaatggaagaatttttaactcaggaatctaagaaacatgaaaatgaatttaatgaagaagtggccttgacagaaatttacaaatacatcgtaaaatattttgatgagattctaaataaactagaaagagaacgagggctggaagaagctcagaaacaactcttgcatgtaaaagtcttatttgatgaaaagaagaaatgcaagtggatgtaagcactctggggcctggcttaatctggcaaagttcttcagacgacttgggagcaaaatggctgcttgagctactctgtgtcgttaatttgttgtttgcacataggttccactttgggcactgtctttttaagagaccaaggcacatgcacagcttttagaaagcaa

SEQ ID NO: 2 - protein, 1568 aa, Artificial Sequence
  Description of Artificial Sequence; note = synthetic construct
MEVSRRKAPPRPPRPAAPLPLLAYLLALAAPGRGADEPVWRSEQAIGAIAASQEDGVFVASGSCLDQLDYSLEHSLSRLYRDQAGNCTEPVSLAPPARPRPGSSFSKLLLPYREGAAGLGGLLLTGWTFDRGACEVRPLGNLSRNSLRNGTEVVSCHPQGSTAGVVYRAGRNNRWYLAVAATYVLPEPETASRCNPAASDHDTAIALKDTEGRSLATQELGRLKLCEGAGSLHFVDAFLWNGSIYFPYYPYNYTSGAATGWPSMARIAQSTEVLFQGQASLDCGHGHPDGRRLLLSSSLVEALDVWAGVFSAAAGEGQERRSPTTTALCLFRMSEIQARAKRVSWDFKTAESHCKEGDQPERVQPIASSTLIHSDLTSVYGTVVMNRTVLFLGTGDGQLLKVILGENLTSNCPEVIYEIKEETPVFYKLVPDPVKNIYIYLTAGKEVRRIRVANCNKHKSCSECLTATDPHCGWCHSLQRCTFQGDCVHSENLENWLDISSGAKKCPKIQIIRSSKEKTTVTMVGSFSPRHSKCMVKNVDSSRELCQNKSQPNRTCTCSIPTRATYKDVSVVNVMFSFGSWNLSDRFNFTNCSSLKECPACVETGCAWCKSARRCIHPFTACDPSDYERNQEQCPVAVEKTSGGGRPKENKGNRTNQALQVFYIKSIEPQKVSTLGKSNVIVTGANFTRASNITMILKGTSTCDKDVIQVSHVLNDTHMKFSLPSSRKEMKDVCIQFDGGNCSSVGSLSYIALPHCSLIFPATTWISGGQNITMMGRNFDVIDNLIISHELKGNINVSEYCVATYCGFLAPSLKSSKVRTNVTVKLRVQDTYLDCGTLQYREDPRFTGYRVESEVDTELEVKIQKENDNFNISKKDIEITLFHGENGQLNCSFENITRNQDLTTILCKIKGIKTASTIANSSKKVRVKLGNLELYVEQESVPSTWYFLIVLPVLLVIVIFAAVGVTRHKSKELSRKQSQQLELLESELRKEIRDGFAELQMDKLDVVDSFGTVPFLDYKHFALRTFFPESGGFTHIFTEDMHNRDANDKNESLTALDALICNKSFLVTVIHTLEKQKNFSVKDRCLFASFLTIALQTKLVYLTSILEVLTRDLMEQCSNMQPKLMLRRTESVVEKLLTNWMSVCLSGFLRETVGEPFYLLVTTLNQKINKGPVDVITCKALYTLNEDWLLWQVPEFSTVALNVVFEKIPENESADVCRNISVNVLDCDTIGQAKEKIFQAFLSKNGSPYGLQLNEIGLELQMGTRQKELLDIDSSSVILEDGITKLNTIGHYEISNGSTIKVFKKIANFTSDVEYSDDHCHLILPDSEAFQDVQGKRHRGKHKFKVKEMYLTKLLSTKVAIHSVLEKLFRSIWSLPNSRAPFAIKYFFDFLDAQAENKKITDPDVVHIWKTNSLPLRFWVNILKNPQFVFDIKKTPHIDGCLSVIAQAFMDAFSLTEQQLGKEAPTNKLLYAKDIPTYKEEVKSYYKAIRDLPPLSSSEMEEFLTQESKKHENEFNEEVALTEIYKYIVKYFDEILNKLERERGLEEAQKQLLHVKVLFDEKKKCKWM

SEQ ID NO: 3 - DNA, 2661 bp, Artificial Sequence
  Description of Artificial Sequence; note = synthetic construct
gctcccggggccacgggatgacgcctcctccgcccggacgtgccgcccccagcgcaccgcgcgcccgcgtccctggcccgccggctcggttggggcttccgctgcggctgcggctgctgctgctgctctgggcggccgccgcctccgcccagggccacctaaggagcggaccccgcatcttcgccgtctggaaaggccatgtagggcaggaccgggtggactttggccagactgagccgcacacggtgcttttccacgagccaggcagctcctctgtgtgggtgggaggacgtggcaaggtctacctctttgacttccccgagggcaagaacgcatctgtgcgcacggtgaatatcggctccacaaaggggtcctgtctggataagcgggactgcgagaactacatcactctcctggagaggcggagtgaggggctgctggcctgtggcaccaacgcccggcaccccagctgctggaacctggtgaatggcactgtggtgccacttggcgagatgagaggctacgcccccttcagcccggacgagaactccctggttctgtttgaaggggacgaggtgtattccaccatccggaagcaggaatacaatgggaagatccctcggttccgccgcatccggggcgagagtgagctgtacaccagtgatactgtcatgcagaacccacagttcatcaaagccaccatcgtgcaccaagaccaggcttacgatgacaagatctactacttcttccgagaggacaatcctgacaagaatcctgaggctcctctcaatgtgtcccgtgtggcccagttgtgcaggggggaccagggtggggaaagttcactgtcagtctccaagtggaacacttttctgaaagccatgctggtatgcagtgatgctgccaccaacaagaacttcaacaggctgcaagacgtcttcctgctccctgaccccagcggccagtggagggacaccagggtctatggtgttttctccaacccctggaactactcagccgtctgtgtgtattccctcggtgacattgacaaggtcttccgtacctcctcactcaagggctaccactcaagccttcccaacccgcggcctggcaagtgcctcccagaccagcagccgatacccacagagaccttccaggtggctgaccgtcacccagaggtggcgcagagggtggagcccatggggcctctgaagacgccattgttccactctaaataccactaccagaaagtggccgtccaccgcatgcaagccagccacggggagacctttcatgtgctttacctaactacagacaggggcactatccacaaggtggtggaaccgggggagcaggagcacagcttcgccttcaacatcatggagatccagcccttccgccgcgcggctgccatccagaccatgtcgctggatgctgagcggaggaagctgtatgtgagctcccagtgggaggtgagccaggtgcccctggacctgtgtgaggtctatggcgggggctgccacggttgcctcatgtcccgagacccctactgcggctgggaccaaggccgctgcatctccatctacagctccgaacggtcagtgctgcaatccattaatccagccgagccacacaaggagtgtcccaaccccaaaccagacaaggccccactgcagaaggtttccctggccccaaactctcgctactacctgagctgccccatggaatcccgccacgccacctactcatggcgccacaaggagaacgtggagcagagctgcgaacctggtcaccagagccccaactgcatcctgttcatcgagaacctcacggcgcagcagtacggccactacttctgcgaggcccaggagggctcctacttccgcgaggctcagcactggcagctgctgcccgaggacggcatcatggccgagcacctgctgggtcatgcctgtgccctggccgcctccctctggctgggggtgctgcccacactcactcttggcttgctggtccactagggcctcccgaggctgggcatgcctcaggcttctgcagcccagggcactagaacgtctcacactcagagccggctggcccgggagctccttgcctgccacttcttccaggggacagaataacccagtggaggatgccaggcctggagacgtccagccgcaggcggctgctgggccccaggtgcgcacggatggtgaggggctgagaatgagggcaccgactgtgaagctggggcatcgatgacccaagactttatcttctggaaaatatttttcagactccctcaaacttgactaaatgcagcgatgctcccagcccaagagcccatgggtcggggagtgggtttggataggagagctgggactccatctcgaccctggggctgaggcctgagtccttctggactcttggtacccacattgcctccttcccctccctctctcatggctgggtggctggtgttcctgaagacccagggctaccctctgtccagccctgtcctctgcagctccctctctggtcctgggtcccacaggacagccgccttgcatgtttattgaaggatgtttgctttccggacggaaggacggaaaaagctctatttttatgttaggcttatttcatgtatagctacttccgactgcc

SEQ ID NO: 4 - protein, 666 aa, Artificial Sequence
  Description of Artificial Sequence; note = synthetic construct
MTPPPPGRAAPSAPRARVPGPPARLGLPLRLRLLLLLWAAAASAQGHLRSGPRIFAVWKGHVGQDRVDFGQTEPHTVLFHEPGSSSVWVGGRGKVYLFDFPEGKNASVRTVNIGSTKGSCLDKRDCENYITLLERRSEGLLACGTNARHPSCWNLVNGTVVPLGEMRGYAPFSPDENSLVLFEGDEVYSTIRKQEYNGKIPRFRRIRGESELYTSDTVMQNPQFIKATIVHQDQAYDDKIYYFFREDNPDKNPEAPLNVSRVAQLCRGDQGGESSLSVSKWNTFLKAMLVCSDAATNKNFNRLQDVFLLPDPSGQWRDTRVYGVFSNPWNYSAVCVYSLGDIDKVFRTSSLKGYHSSLPNPRPGKCLPDQQPIPTETFQVADRHPEVAQRVEPMGPLKTPLFHSKYHYQKVAVHRMQASHGETFHVLYLTTDRGTIHKVVEPGEQEHSFAFNIMEIQPFRRAAAIQTMSLDAERRKLYVSSQWEVSQVPLDLCEVYGGGCHGCLMSRDPYCGWDQGRCISIYSSERSVLQSINPAEPHKECPNPKPDKAPLQKVSLAPNSRYYLSCPMESRHATYSWRHKENVEQSCEPGHQSPNCILFIENLTAQQYGHYFCEAQEGSYFREAQHWQLLPEDGIMAEHLLGHACALAASLWLGVLPTLTLGLLVH

SEQ ID NO: 5 - DNA, 18 bp, Artificial Sequence
  Description of Artificial Sequence; note = synthetic construct
aaccattgcactgcaacc

SEQ ID NO: 6 - DNA, 22 bp, Artificial Sequence
  Description of Artificial Sequence; note = synthetic construct
gattccatcttcaagaatcacg

SEQ ID NO: 7 - DNA, 21 bp, Artificial Sequence
  Description of Artificial Sequence; note = synthetic construct
cacgcacgatttcccgctcgg

SEQ ID NO: 8 - DNA, 20 bp, Artificial Sequence
  Description of Artificial Sequence; note = synthetic construct
caggctgtgctatcctgtac

(19) United States
(12) Patent Application Publication
(10) Pub. No.: US 2011/0158953 A1
(43) Pub. Date: Jun. 30, 2011

(54) TUMOR SUPPRESSION THROUGH PLEXIN C1

(75) Inventor: Glynis Scott, Rochester, NY (US)

(73) Assignee: UNIVERSITY OF ROCHESTER, Rochester, NY (US)

(21) Appl. No.: 12/999,823

(22) PCT Filed: Jun. 17, 2009
(86) PCT No.: PCT/US09/47597

§ 371 Date: Mar. 7, 2011

Related U.S. Application Data
(62) Division of application No. 61/073,352, filed on Jun. 17, 2008.

(51) Int. Cl. A61K 38/16; C12Q 1/02; C12Q 1/68; G01N 33/53; C40B 30/00; A61K 35/76; A61P 35/00

(52) U.S. Cl. 424/93.6; 435/29; 435/6.1; 435/6.12; 435/7.92; 506/7; 435/7.1; 514/19.8

(57) ABSTRACT

Disclosed are compositions and methods relating to Plexin C1 and diagnosing, treating, and evaluating treatment for cancer.

This application claims the benefit of U.S. Provisional Application No. 61/073,352, filed on Jun. 17, 2008, which is incorporated herein by reference in its entirety.

This invention was made with government support under Grant AR045427 awarded by the National Institutes of Health. The government has certain rights in the invention.

### I. BACKGROUND

Melanoma is a deadly skin cancer that, through multiple mechanisms, arises from melanocytes or melanocyte stem cells. De novo expression of growth factors and constitutive activation or up-regulation of receptor expression, stimulate pathways that result in melanoma initiation and progression (Haass et al., 2005; Hamoen et al., 2001; Hess et al., 2005; Klein et al., 1991). However, to date, there has been no way to quickly determine how severe and pervasive a melanoma was. Moreover, reducing progression of melanoma remains a goal for melanoma treatment.

### II. SUMMARY

Disclosed are methods and compositions related to diagnosing, evaluating, and treating cancer. Also disclosed are methods and compositions related to diagnosing, evaluating, and treating melanomas.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows that Plexin C1 protein expression is decreased in human melanoma. Total cell lysates of melanocytes (MC) and 6 human melanoma cell lines were resolved on 7.5% SDS-PAGE and blotted with antibodies to Plexin C1. The lower part of the membrane was blotted for β-actin. Melanocytes express large amounts of Plexin C1. Plexin C1 is completely absent in cell lines WM115 and C32, is decreased in WM165, YURIF and YUSIK, and is slightly increased in YUMAC. Results are representative of 3 separate experiments.

FIG. 1B shows that Plexin C1 message is decreased or absent in 5/6 human melanoma cell lines. Message RNA was reverse transcribed from melanocytes and six human melanoma cell lines. Quantitative real-time PCR of each sample was performed in triplicate against a standard curve of Plexin C1 PCR product to arrive at a quantitative Plexin C1 level. Four of the 6 cell lines either had no Plexin mRNA (C32 and WM115) or decreased Plexin C1 mRNA (YUSIK and WW165) compared with melanocytes. Plexin C1 message was increased compared with melanocytes in 2 cell lines (YURIF and YUMAC). Results are the averaged amount of Plexin C1 message (+/−SD) of three separate experiments.

FIGS. 1C and D show that DNA hypermethylation regulates Plexin C1 expression in melanoma cells. Melanoma cells were treated with the DNA methylation inhibitor 5-AzaDc (“+” 3 or vehicle (“−”) for 5 days and total cell lysates were resolved on 7.5% SDS-PAGE and blotted for Plexin C1. An increase in Plexin C1 protein was seen in YUMAC, whereas a decrease in Plexin C1 protein was seen in YURIF and WW165 melanoma cells (arrows; FIG. 1C). Results are representative of 2 separate experiments. 1D) Treatment of YUMAC, YURIF and WW165 melanoma cells with 5-AzaDC for 5 days at doses ranging from 0.1 μM to 3.0 μM showed that a dose of 0.1 μM was sufficient to increase Plexin C1 expression in YUMAC cells, and that YURIF and WW165 expression was increased at a dose of 1.0 μM (arrows). Results are representative of 2 separate experiments.

FIG. 2 shows that loss of Plexin C1 is correlated with progression of primary melanoma to metastatic melanoma. Compiled data of Plexin C1 intensity scores (IS) of TMAs of nevi, melanoma primary to the skin, and metastatic melanoma. Shown is the percentage of cases of nevi, melanoma and metastatic melanoma with no Plexin C1 staining, moderately strong staining, and strong staining. Sixty-six percent of metastatic melanomas (pale gray bars) did not express Plexin C1, whereas all nevi (dark gray bars) showed either moderate or strong expression of Plexin C1. Expression of Plexin C1 was significantly different in nevi compared with metastatic melanoma, and between primary melanoma (black bars) and metastatic melanoma (p<0.001). Nevi expressed more Plexin C1 than primary melanoma, and these differences were statistically significant (p<0.05).

FIG. 3A shows that benign Nevi Strongly Express Plexin C1. Two representative cases of benign nevi from TMA stained for Plexin C1 are shown. One core from each case, with a high power inset, is shown, along with the assigned intensity score (IS). The percentage of all cases with IS of 0-1.5 (no staining), 1.6-2.5 (moderate staining) and 2.6-3.0 (strong staining) is presented in the accompanying graph. The nevic cells in virtually all nevi showed moderate to strong expression of Plexin C1.

FIG. 3B shows that primary melanomas show Plexin C1 expression intermediate between nevi and metastatic melanoma. Four representative cases of melanomas stained for Plexin C1 from TMA are shown. One core from each case, with a high power inset, along with the assigned intensity score (IS) is shown. The percentage of all cases with IS of 0-1.5 (no staining), 1.6-2.5 (moderate staining) and 2.6-3.0 (strong staining) is presented in the accompanying graph. Of note, one case showed melanoma arising within a nevus. In this case, strong expression of Plexin C1 within the nevic cells (*) is present, juxtaposed with melanoma cells with weaker Plexin C1 expression (arrows).

FIG. 3C shows that Plexin C1 expression decreases with increasing depth of invasion. When cases were separated into groups of different depths of invasion, a clear trend of decreasing Plexin C1 expression with increasing depth of invasion is present. Thin melanomas (0.1-1 mm) had a significantly higher IS for Plexin C1 compared with melanomas 4 mm or thicker (IS of 3 and 1.33 respectively, p<0.001).

FIG. 3D shows that metastatic melanomas express low levels of Plexin C1. Four representative cases of metastatic melanoma from TMA stained for Plexin C1 are shown. One core from each case, with a high power inset, along with the assigned intensity score (IS) is shown. The percentage of all cases with IS of 0-1.5 (no staining), 1.6-2.5 (moderate staining) and 2.6-3.0 (strong staining) is presented in the accompanying graph. The vast majority of metastatic melanomas did not express Plexin C1.

FIG. 4A shows that Sema7A stimulates the phosphorylation of FAK and cofilin in human melanocytes. Melanocytes were placed in basal medium (MCDB-153) for 8 hours and were treated with recombinant Sema7A (100 ng/ml) for the indicated time points; controls were treated with vehicle. Total cell lysates were resolved on 10% SDS-PAGE and blotted for phosphorylated focal adhesion kinase (FAK) and phosphorylated cofilin. Sema7A stimulated the rapid (within 5 min) phosphorylation of FAK, indicative of integrin activation. Levels of inactive (phosphorylated) cofilin were also rapidly increased in melanocytes upon Sema7A treatment, which peaked at 15 min. Results are representative of 3 separate experiments performed on pooled cultures of human melanocytes.

FIG. 4B shows that a dose response analysis of FAK and cofilin phosphorylation in response to Sema7A was performed. Melanocytes were placed in basal medium (MCDB-153) for 8 hours and were treated with recombinant Sema7A at doses ranging from 1 ng/ml to 100 ng/ml, for 5 min. Controls were treated with vehicle. Total cell lysates were resolved on 10% SDS-PAGE and blotted for phosphorylated FAK and phosphorylated cofilin. Maximal FAK and cofilin phosphorylation was observed at 50 ng/ml (arrow), and in the case of FAK, occurred at concentrations of Sema7A as low as 1 ng/ml (asterick). Cofilin phosphorylation was first detected at a dose of 10 ng/ml of Sema7A. Results are representative of 2 separate experiments performed on pooled cultures of human melanocytes.

FIGS. 4C and 4D show that Sema7A stimulates MAP kinase activation in melanocytes: Melanocytes were placed in basal medium (MCDB-153) for 8 hours and were treated for 5 min with recombinant Sema7A at doses ranging from 1 ng/ml to 50 ng/ml; controls were treated with vehicle. Total cell lysates were resolved on 10% SDS-PAGE and blotted for phosphorylated Erk1/Erk2; the blot was stripped and re-blotted for total Erk1/Erk2. A dose-dependent increase in P-Erk1/Erk2 was observed in response to Sema7A, with a peak response seen at a dose of 25 ng/ml (arrow). Results are representative of 2 separate experiments. The specificity of MAP kinase activation was tested in melanocytes by the inclusion of the selective p42/44 MAP kinase inhibitor PD 98059 (10 μM) in cells treated with Sema7A (25 ng/ml) for 5 minutes (FIG. 4D). Shown are cell lyates resolved on SDS-PAGE and blotted for P-Erk1/Erk2. The blot was stripped and reblotted for total Erk1/Erk2. As expected, Sema7A stimulated phosphorylation of Erk1/Erk2 within 5 minutes of treatment. Cells treated with PD 98059 for one hour prior to treatment with Sema7A showed no induction of Erk1/Erk2 phosphorylation. Results are representative of 2 separate experiments.

FIGS. 5A and B show that silencing of Plexin C1 abrogates cofilin phosphorylation/inactivation, but has no effect on MAP kinase activation. Melanocytes were transfected with siRNAs to Plexin C1 (Si) or scrambled siRNAs (Sc) and 48 hours later cells were placed in basal medium for 8 hours and then treated for 5 or 15 minutes with Sema7A (50 ng/ml). Total cell lysates were resolved on 7.5% SDS-PAGE. The top of the membrane was blotted for Plexin C1, and the bottom was blotted for phospho-cofilin (FIG. 5A). Phosphorylation/inactivation of cofilin, in response to Sema7A, correlated with levels of Plexin C1 expression; at the 5 minute time point, in which Plexin C1 was silenced >50%, there was a >50% reduction in phosphorylated cofilin. In the 15 minute sample, in which total silencing of Plexin C1 was achieved, there was no detectable phosphorylation of cofilin. Results are representative of 3 separate experiments. B) Melanocytes were transfected with siRNAs to Plexin C1 (Si) or scrambled siRNAs (Sc) as described above and treated for 5 minutes with Sema7A (25 ng/ml). Total cell lysates were resolved on 7.5% SDS-PAGE. The top of the membrane was blotted for Plexin C1, and the bottom was blotted for phospho-Erk1/2. Knockdown of Plexin C1 did not affect phosphorylation of Erk1/2 in response to Sema7A. Results are representative of 3 separate experiments. FIG. 5C shows that human melanocytes and melanoma cells express LIMKII. Lysates (40 μg) of human melanocytes (MC), WM115, WW165, C32, YURIF, YUMAC and YUSIK were resolved on 10% SDS-PAGE and blotted with antibodies that recognize LIMKI or LIMKII. None of the cells express LIMKI. Melanocytes, and 3/5 melanoma cell lines, express LIMK II. Interestingly, the expression of LIMKII correlates with Plexin C1 expression (See FIG. 1A). Results are representative of 2 separate experiments.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Using the Compositions

### 1. Methods of Determining the Prognosis of a Disease

Plexin C1 is disclosed herein as having decreased expression and/or activity levels in metastatic cancers including, for example, metastatic melanoma. Accordingly, disclosed herein are methods of determining the prognosis of a cancer in a subject comprising obtaining a tissue sample from the subject and measuring the level of Plexin C1 in the sample, wherein a decrease in the level of Plexin C1 relative to a normal control indicates a more aggressive cancer.

Plexins are a family of transmembrane receptors that bind to secreted and membrane bound semaphorins (Artigiani et al., 1999; Tamagnone et al., 1999). Plexins were identified through their homology to the extracellular domain of the scatter factor receptors, and the cytoplasmic domains of Plexins are highly conserved. Some of the effects of Plexin signaling are due to binding of GTP-binding proteins, to inhibition of integrin, and to cofilin activation (Pasterkamp, 2005; Swiercz et al., 2004; Walzer et al., 2005a; Walzer et al., 2005b). Data indicate that Plexin C1 is a receptor for Sema7A in some, but not all cell types, indicating the existence of additional ligands for Plexin C1 (Pasterkamp et al., 2003; Walzer et al., 2005a; Walzer et al., 2005b). There is very limited information on the signaling pathways stimulated by Plexin C1; however, in murine dendritic cells, Plexin C1 signaling inactivates cofilin and inhibits integrins, resulting in decreased adhesion and migration (Walzer et al., 2005a). Recent studies showed that human melanocytes express abundant Plexin C1 at the message and protein level, and that Plexin C1 is expressed in normal melanocytes in the skin in vivo (Scott et al., 2008). The functional significance of Plexin C1 in mediating Sema7A effects in melanocytes was demonstrated through experiments in which the silencing of Plexin C1 resulted in enhanced adhesion and spreading in response to Sema7A. While the mechanisms have not been fully determined, Plexin C1 signaling abrogates melanocyte adhesion (similar to murine dendritic cells).

Melanoma is a deadly skin cancer that, through multiple mechanisms, arises from melanocytes or melanocyte stem cells. De novo expression of growth factors and constitutive activation or up-regulation of receptor expression, stimulate pathways that result in melanoma initiation and progression (Haass et al., 2005; Hamoen et al., 2001; Hess et al., 2005; Klein et al., 1991). Semaphorins regulate cell migration and motility in virtually all cell types examined, so there is keen interest in the possible role of semaphorins in tumor progression (Artigiani et al., 1999; Bielenberg and Klagsbrun, 2007; Hu et al., 2007; Kreuter et al., 2002). Investigation of the potential role of semaphorins in melanoma progression is limited; however, semaphorin 3F was identified as a tumor suppressor of melanoma through effects on melanoma proliferation (Bielenberg et al., 2004). Here, these experiments demonstrate for the first time that Plexin C1 expression is lost during melanoma invasion and metastasis. These experiments further demonstrate that cofilin, an actin binding protein involved in actin assembly, is a downstream target of Plexin C1 signaling in melanocytes. Plexin C1 is a novel tumor suppressor protein for melanoma progression, and that loss of Plexin C1 promotes melanoma progression through unopposed cofilin activation.

It is further understood that understanding the level of Plexin C1 in a subject also provides a manner by which the aggressiveness or metastatic potential of a cancer can be determined, wherein in a decreased level of Plexin C1 indicates an aggressive cancer or a cancer with high metastatic potential. Thus, for example, disclosed herein are methods of characterizing the metastatic potential of a cancer comprising obtaining a tissue sample from the subject and measuring the level of Plexin C1 in the sample, wherein a decrease in the level of Plexin C1 relative to a normal control indicates a metastatic cancer. Also disclosed are methods of assessing the aggressiveness of a cancer comprising obtaining a tissue sample from the subject and measuring the level of Plexin C1 in the sample, wherein a decrease in the level of Plexin C1 relative to a normal control indicates a more aggressive cancer.

It is understood and herein contemplated that cancer progression and depth of invasion are significant indicators of the severity of a disease. Moreover, it is understood that monitoring the progression of depth of invasion of a cancer provides an indication of the efficacy of a treatment as well as the as how aggressive a treatment is needed to treat a subject suffering from a cancer. Therefore, disclosed herein are methods of assessing cancer progression comprising obtaining a tissue sample from a subject and measuring the level of Plexin C1 in the sample, wherein a decrease in the level of Plexin C1 relative to a normal control indicates an increased progression of the cancer. Also disclosed are methods of assessing the depth of invasion of a cancer comprising obtaining a tissue sample from a subject and measuring the level of Plexin C1 in the sample, wherein a decrease in the level of Plexin C1 relative to a normal control indicates an increased depth of invasion. Also disclosed herein are methods of determining the aggressiveness of a treatment of a cancer comprising obtaining a tissue sample from the subject and measuring the level of Plexin C1 in the sample, wherein a decrease in the level of Plexin C1 relative to a normal control indicates a more aggressive treatment should be used.

It is understood that Plexin C1 expression can be measured by measuring expression at the nucleic acid level, protein level, or measuring the effects of Plexin C1 activity. It is further understood and herein contemplated that the level of Plexin C1 in a sample can be determined by any method known in the art for the determination of expression or activities levels. Therefore, it is contemplated herein that Plexin C1 levels can be measured by protein array, immunohistochemical staining, Western blot, flow cytometry, ELISA, ELISPOT, Quantitative PCT, and microarrray.

### 2. Immunodetection

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); 1Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyde Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab−Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab−Ag]n), and reagent antigens are used to detect specific antibody ([Ab−Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log10MW for known samples, and read off the logMr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods.

Laemmli, U. K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunospot Assay (ELISPOT is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. For descriptions of ELISA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995;U.S. Pat. No. 4,376,110, each of which is incorporated herein by reference in its entirety and specifically for teachings regarding ELISA methods.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™ SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### 3. Method of Assessing the Biologic Behavior of Melanoma and Potential for Melanoma Progression

The disclosed compositions can be used to diagnose or assess the progression/measure the metastatic potential any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### 4. Method of Treating Cancer

The disclosed compositions can be used to treat any disease where uncontrolled cellular proliferation occurs such as cancers. It is understood and herein contemplated that one such method of treating a cancer can be accomplished by inhibiting factors that aid in metastises such as actin assembly. Thus, disclosed herein are methods of treating a cancer comprising administering to the subject an agent that inhibits actin assembly. It is further understood that disclosed herein are methods of inhibiting a metastatic progression of a disease or cancer progression (e.g., melanoma progression) in a subject comprising administering to the subject an agent that inhibits actin assembly.

It is understood and disclosed herein that cofilin activation causes actin assembly in metastatic cells. Accordingly, one method of treating a cancer is by administering to a subject with a cancer is by administering to the subject an agent that inhibits cofilin. One method by which cofilin activation occurs is through phosphorylation of Ser33. Therefore, disclosed herein are methods of inhibiting cofilin activation comprising administering to the subject an agent that phosphorylates cofilin. It is further disclosed herein that cofilin phosphorylation and activation is the result of Plexin C1 activity. Therefore disclosed herein are methods of treating cancer, inhibiting the metastatic progression of a cancer, and/or inhibiting cancer progression in a subject comprising administering to a subject Plexin C1. It is further understood that Plexin C1 can inhibit metastatic progression through means other than inhibition of actin assembly. Accordingly, disclosed herein are methods of inhibiting the metastatic progression of a cancer in a subject comprising administering to the subject an agent that increases Plexin C1 expression or signaling. One means to increase Plexin C1 expression or signaling or to inhibit actin assembly is through the administration of exogenous Plexin C1. Thus, disclosed herein are methods of inhibiting actin assembly or increasing Plexin C1 expression or signaling comprising administering to the subject Plexin C1. It is understood and herein contemplated that Plexin C1 can be administered through any means known in the art, such as, for example, by administering a viral vector comprising Plexin C1. It is further understood the Sema7A can increase Plexin C1 expression or signaling. Thus, also disclosed are methods of increasing Plexin C1 expression or signaling wherein the agent that increases Plexin C1 signalling is Sema 7a.

It is also understood that activation or inhibition of additional pathways of a mestatic cell can aid treatment. Therefore, disclosed herein are methods of treating cancer, inhibiting the metastatic progression of a cancer, and/or inhibiting cancer progression further comprising administering to the subject an agent that activates focal adhesion kinase (FAK). For example, disclosed herein are methods of treatment further comprising the administration of Sema7A.

The disclosed methods of treating can be used with any uncontrolled proliferative disease such as a mestatic cancer. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### 5. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

### 6. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as Plexin C1 or Sema7A into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed Plexin C1 and Sema7A plasmids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 7. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Plexin C1 or Sema7A is disclosed and discussed and a number of modifications that can be made to a number of molecules including the Plexin C1 or Sema7A are discussed, specifically contemplated is each and every combination and permutation of Plexin C1 or Sema7A and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Homology/Identity

It is understood that one way to define any known variants and derivatives or those that might arise, of the disclosed genes and proteins herein is through defining the variants and derivatives in terms of homology to specific known sequences. For example SEQ ID NO: 1 sets forth a particular sequence of an Plexin C1 gene and SEQ ID NO: 2 sets forth a particular sequence of the protein encoded by SEQ ID NO: 1, an Plexin C1 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Peptides

### a) Protein Variants

As discussed herein there are numerous variants of the Plexin C1 protein and Sema7A protein that are known and herein contemplated. In addition, to the known functional Plexin C1 and Sema7A strain variants there are derivatives of the Plexin C1 and Sema7A proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of a Plexin C1 gene and SEQ ID NO: 2 sets forth a particular sequence of a Plexin C1 protein. Specifically disclosed are variants of these and other proteins herein disclosed which have at least, 70% or 75% or 80% or 85% or 90% or 95% identity to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 2 is set forth in SEQ ID NO: 1.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### 3. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with Plexin C1. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### 4. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing an a cancer such as, for example, metastatic melanoma, the efficacy of the therapeutic antibody can be assessed in various ways well known to the skilled practitioner. For instance, one of ordinary skill in the art will understand that a composition, such as an antibody, disclosed herein is efficacious in treating or inhibiting an a cancer such as, for example, metastatic melanoma, in a subject by observing that the composition reduces depth of invasion of a cancer or prevents a further increase in cancer progression.

The disclosed compositions and methods can also be used for example as tools to isolate and test new drug candidates for a variety of cancers or diseases with uncontrolled cellular proliferation.

### 5. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

### 6. Compositions Identified by Screening with Disclosed Compositions/Combinatorial Chemistry

### a) Combinatorial Chemistry 183. The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed herein, such as Plexin C1 or cofilin, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function.

It is understood that the disclosed methods for identifying molecules that inhibit Cofilin (for e.g., by phosphorylating Cofilin at Ser33) or activate Plexin C1 can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23) 12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23) 12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice is attached to a DNA binding domain of a transcriptional activation protein, such as Gal 4. By performing the Two-hybrid technique on this type of system, molecules that bind Cofilin can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. No. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

### 7. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include a first agent that specifically binds to Plexin C1, one or more reagents for detecting the presence of the first agent, and a control. For example, disclosed is a kit for assessing a subject's risk for acquiring metastatic melanoma. Also disclosed herein are kits for measuring Plexin C1 levels in a cancer in a subject comprising a first agent that specifically binds to Plexin C1, one or more reagents for detecting the presence of the first agent, and a control. It is understood and herein contemplated that the first agent can be an antibody. For example, the first angent can be an antibody that binds Plexin C1. It is further understood that the kit can comprise any reagents necessary for direct or indirect detection of the first agent including one or more second agents. In particular, disclosed herein are any reagents disclosed herein that can be used for detection. It is also understood that the first agent antibody can be a labeled antibody including but not limited to biotin, or fluorescent antibodies. Also disclosed are kits including a second agent which binds to the first agent.

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Introduction

Semaphorins are a large class of secreted and membrane anchored proteins that play a critical role in neuronal pathfinding and axon guidance in selected areas of the developing nervous system (Pasterkamp and Verhaagen, 2006). Semaphorins are subdivided into eight subfamilies: two found in invertebrates (Classes 1 and 2), one in viruses (Class 8), and five in vertebrates (Classes 3-7). All semaphorins have a conserved semaphorin (sema) domain in the amino terminus, which domain is also present in their receptors, the Plexins. Furthermore, semaphorins stimulate multiple signaling pathways, and. The prototypical semaphorin, semaphorin 3A/collapsin-1, causes growth cone collapse of sensory neurons through remodeling of cytoskeletal proteins, and the majority of semaphorins described to date act as repellant signals to neurons (Giger et al., 1998; Pasterkamp et al., 1998a; Pasterkamp et al., 1998b; Pasterkamp et al., 1999). While their expression and function was originally described in the brain and spinal cord, semaphorins are now known to be widely expressed and have diverse functions. The protean effects of semaphorins include regulation of blood vessel development, modulation of the immune system, and drug resistance (Ishida et al., 2003; Moretti et al., 2006; Tamagnone and Comoglio, 2000; Yamada et al., 1997). Semaphorins have also been reported to have roles as tumor suppressors and tumor promoters; a role accomplished, in part, through regulation of tumor vessel formation (Basile et al., 2004; Basile et al., 2006; Bielenberg et al., 2004; Kessler et al., 2004).

Lymphoid organs, odontoblasts, bone cells, the nervous system of mice, human epidermal keratinocytes, fibroblasts, and the endothelial cells of blood vessels of the skin express Semaphorin 7A (Sema7A) at high levels (Czopik et al., 2006; Delorme et al., 2005; Koh et al., 2006; Maurin et al., 2005; Scott et al., 2008; Suzuki et al., 2007). Sema7A binds to two distinct classes of receptors, the β1-integrins and Plexin C1 (Pasterkamp et al., 2003; Walzer et al., 2005a; Walzer et al., 2005b). Integrins are transmembrane heterodimeric proteins that link the extracellular matrix with the cytoskeleton and are critical for cell adhesion and migration (Arnaout et al., 2007; Huveneers et al., 2007; Takagi 2007). The cytoplasmic domain of integrins couples to vinculin, talin and paxillin to form the focal adhesion complex. Integrins also bind with kinases such as focal adhesion kinase (FAK), which is phosphorylated upon integrin activation (Matsumoto et al., 1995). Sema7A binds β₁-integrins, resulting in neurite extension, cytokine production, and migration in neurites, monocytes, and bone cells respectively (Delorme et al., 2005; Pasterkamp et al., 2003; Suzuki et al., 2007). By utilizing blocking (β₁-integrin antibodies, and echistatin, a snake venom that inhibits integrins, studies have shown that Sema7A, through the β₁-integrins, stimulates melanocyte adhesion and dendrite formation. (Scott et al., 2008). Therefore, integrin mediated signaling appears to be a common mechanism by which the effects of Sema7A are controlled.

### b) Results

(1) Plexin C1 Expression is Decreased at the Protein and Message Level in Human Melanoma Cell Lines

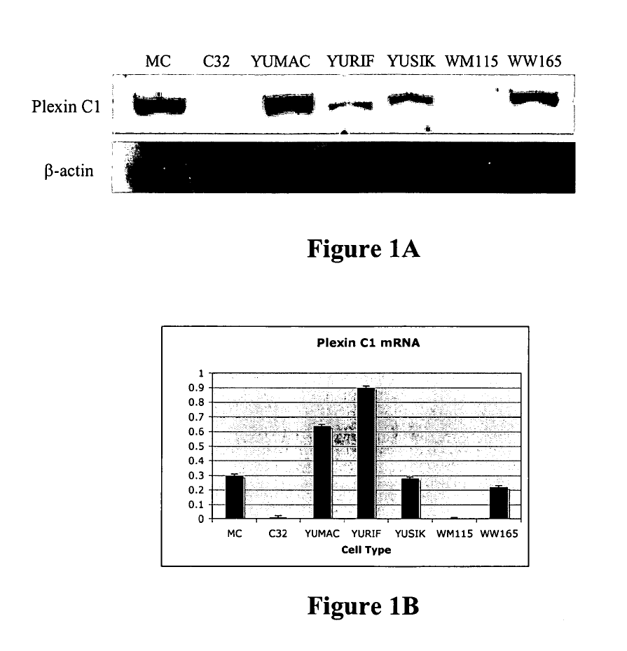

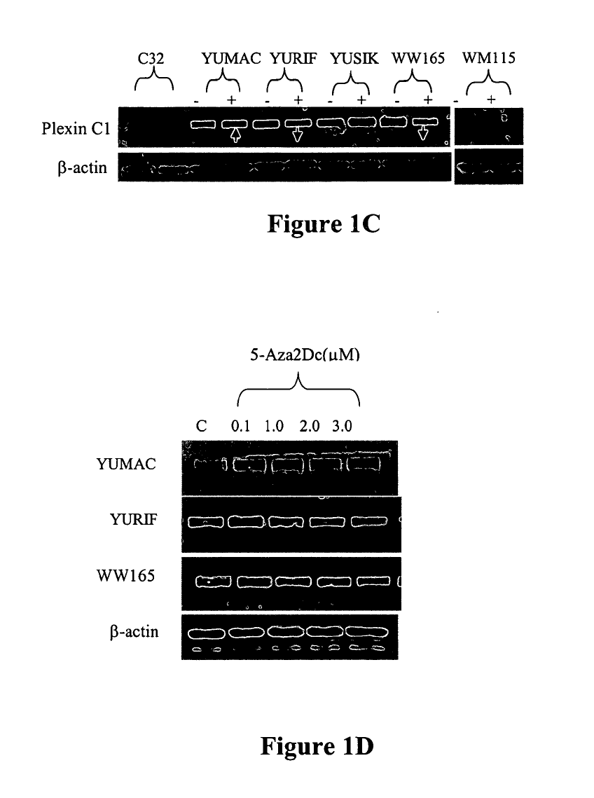

Total cell lysates of 6 human melanoma cell lines and normal human melanocytes were blotted for Plexin C1 (FIG. 1A). Plexin C1 is expressed in normal human melanocytes, is absent from cell lines WM115 and C32, and is decreased in WW165, YURIF and YUSIK. YUMAC show increased Plexin C1 protein expression compared with normal melanocytes. Quantitative real time PCR showed no Plexin C1 PCR product in the two cell lines (WM115 and C32) that completely lacked Plexin C1 protein (FIG. 1B). The YUMAC and YURIF cell lines expressed Plexin C1 PCR product at higher levels than melanocytes. This increase in expression occurred despite a lower Plexin C1 protein level as compared with melanocytes in one of these cell lines (YURIF). The YUSIK and WW165 melanoma cell lines, which had a lower level of Plexin C1 protein expression, also expressed less Plexin C1 mRNA. Because the de novo production of growth factors is implicated in melanoma tumor progression, the cell surface expression of Sema7A was determined by FACs analysis of stained cells. None of the 6 human melanoma cell lines expressed detectable Sema7A.

Promotor hypermethylation plays a major role in transcriptional silencing of tumor suppressor genes in cancer and in melanoma (Dahl and Guldberg, 2007; Gronbaek et al., 2007; Rothhammer and Bosserhoff, 2007). To determine if Plexin C1 expression is regulated by DNA hypermethylation, melanoma cells were treated for 5 days with the DNA methylation inhibitor 5-Aza-2′-Deoxycytidine (5-Aza2Dc; 3 μM). Total cell lysates were then blotted for Plexin C1. Of the 6 melanoma cell lines tested, the C32, WM115 and YUSIK cell lines showed no change in Plexin C1 protein levels. The YUMAC cell line showed an increase in Plexin C1. The WW165 and YURIF cell lines showed a reduction in Plexin C1 protein expression with 5-Aza2Dc treatment, which reduction was most marked in WW165 (FIG. 1C). A dose response analysis showed that, at a dose of 0.1 μM, 5-Aza2Dc increased Plexin C1 expression in the YUMAC cell line. But, a dose of 1.0 μM was the lowest dose that stimulated Plexin C1 expression in the YURIF and WW165 cell lines (FIG. 1C).

(2) Plexin C1 Expression Correlates with Melanoma Progression

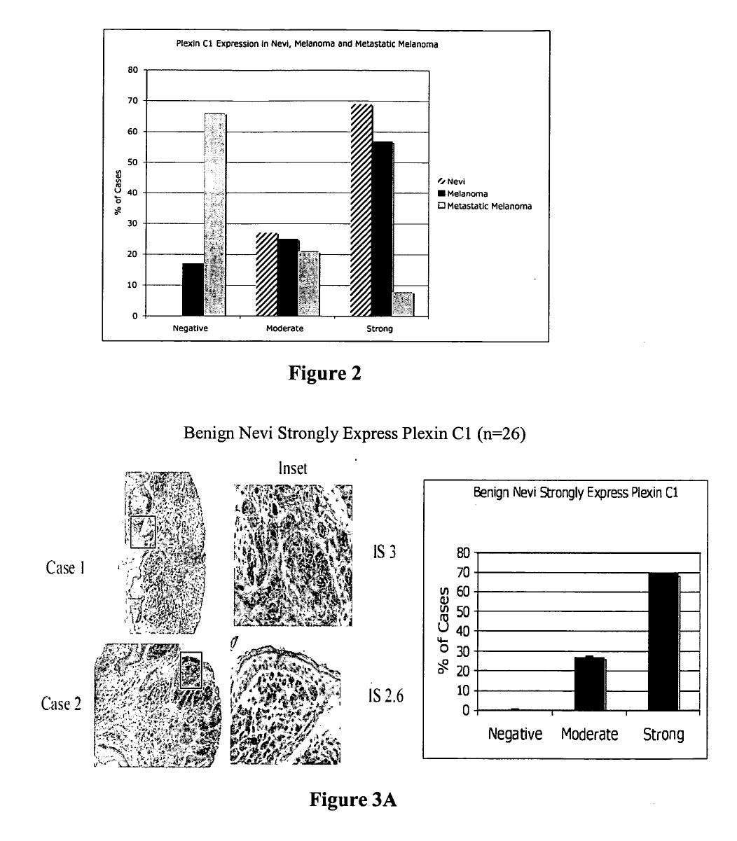

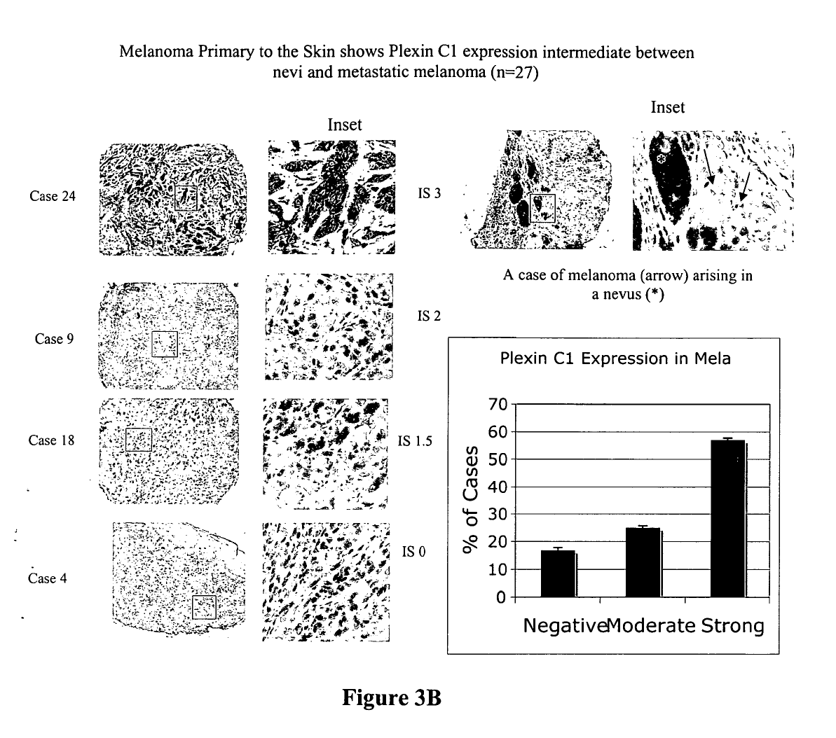

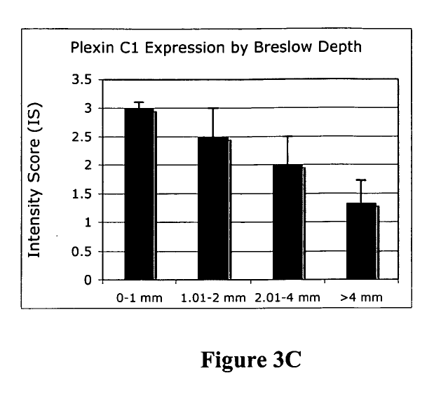

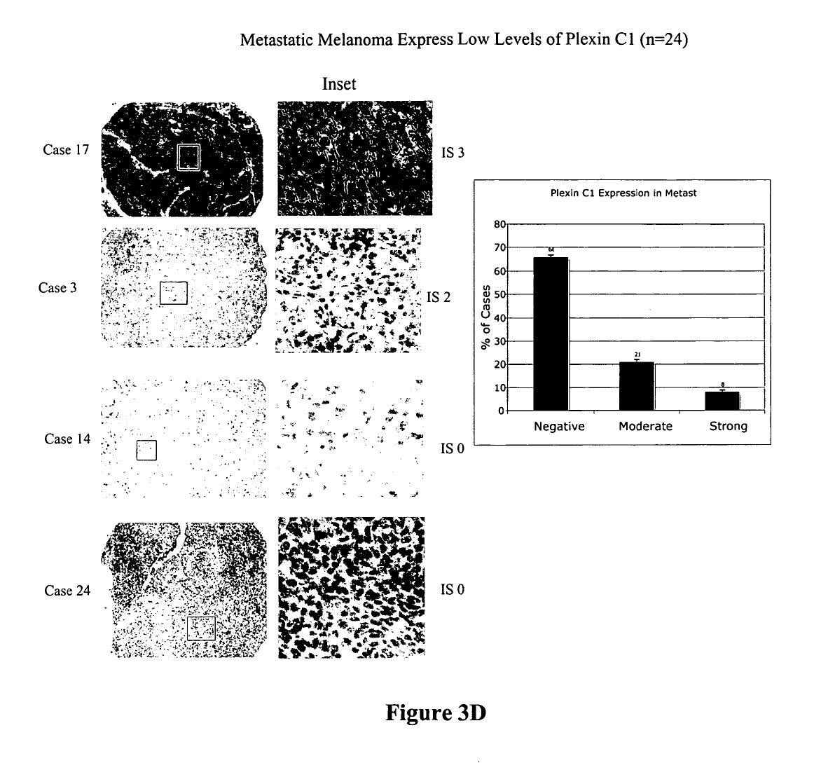

Using 26 cases of benign nevi, 27 cases of melanoma primary to the skin, and 24 cases of metastatic melanoma, tissue microarrays were constructed. FIG. 2 presents the percentage of cases from each group that exhibited no staining (0-1.5 intensity score (IS)), moderate staining (1.6-2.5 IS), and strong staining (2.6-3.0 IS). While all nevi showed either moderate or strong expression of Plexin C1, sixty-six percent of metastatic melanomas did not express Plexin C1. The Plexin C1 expression level in nevi significantly differed from the level in metastatic melanoma. The Plexin C1 expression levels in the primary melanoma and the metastatic melanoma were also significantly different from one another (p<0.001). Nevi expressed more Plexin C1 than primary melanoma, and this differences was also statistically significant (p<0.05). FIG. 3A-FIG. 3D show representative photographs of nevi, melanoma, and metastatic melanoma stained for Plexin C1.

The average IS of the 26 nevi was 2.76 (+/−0.34 SD). 69% of the 26 nevi had strong expression of Plexin C1 (IS between 2.6-3.0), 27% of nevi showed moderately strong Plexin C1 expression (IS between 1.6 and 2.5), and 0% of nevi expressed no Plexin C1 staining (IS between 0 and 1.5). FIG. 3A shows representative cores of 2 cases of nevi that were stained for Plexin C1. Strong and diffuse membranous expression of Plexin C1 is observed. The lowest IS for any nevus was 2.3.

The expression of Plexin C1 by melanoma primary to the skin was more heterogeneous (FIG. 3B). While the IS score of primary melanoma ranged from 0-3, the average IS for all cases (n=27) was 2.38 (+/−0.84 SD). The average IS score for the primary melanoma significantly differed from that of the nevi. (p<0.05). Fifty-seven percent of the primary melanoma cases showed strong staining, 25% showed moderately strong staining, and 17% showed no staining. Of note, a case of melanoma arising within a nevus showed strong staining of the nevic cells, but weak staining of the melanoma (FIG. 3C).

The depth of invasion of melanoma is a major prognostic indicator (Balch et al., 1980; Patrick et al., 2007). When Plexin C1 expression was analyzed in melanomas with different depths of invasion, a trend emerged. Diminished Plexin C1 expression correlated with increased depth of invasion (FIG. 3C). Melanomas with a depth <1 mm (n=9) had a Plexin C1 IS score of 3 (+/−0.10 SD). These melanomas are considered thin melanomas and there is nearly a 100% 5-year survival rate for this type of melanoma. Melanomas with a depth of invasion between 1.01 and 2.0 mm (n=8) had an IS score of 2.5 (+/−0.53 SD). Melanomas with a depth of invasion between 2.01 and 4.0 mm (n=6) had an IS score of 2 (+/−0.51 SD) and melanomas with a depth of invasion of >4 mm (n=4) had an IS of 1.32 (+/−0.47 SD). A melanoma with a depth of invasion >4 mm is considered an advanced tumors, and has only a 50% 5-year survival rate. A Student's t-test showed that the levels of Plexin C1 expression in thin melanomas (depth of invasion <1 mm) and in deeply invasive tumors (depth >4 mm) were different from one another (p<0.001).

A striking decrease in Plexin C1 expression in metastatic melanoma was observed (FIG. 3D). The average IS for Plexin C1 in all cases of metastatic melanoma (n=24) was 1.0 (+/−0.97). This level significantly differed from that of the nevi (p<0.001) and from that of the melanoma primary to the skin (p<0.001). Sixty-seven percent of metastatic melanomas had no Plexin C1 staining, and only 20% of cases showed moderately strong staining. Two of the 5 cases that showed strong Plexin C1 staining (IS 1.6-2.5) were tumors metastatic to skin and bone.

(3) Sema7A Stimulates Cofilin Inactivation, and Activates FAK and MAP Kinase, in Human Melanocytes

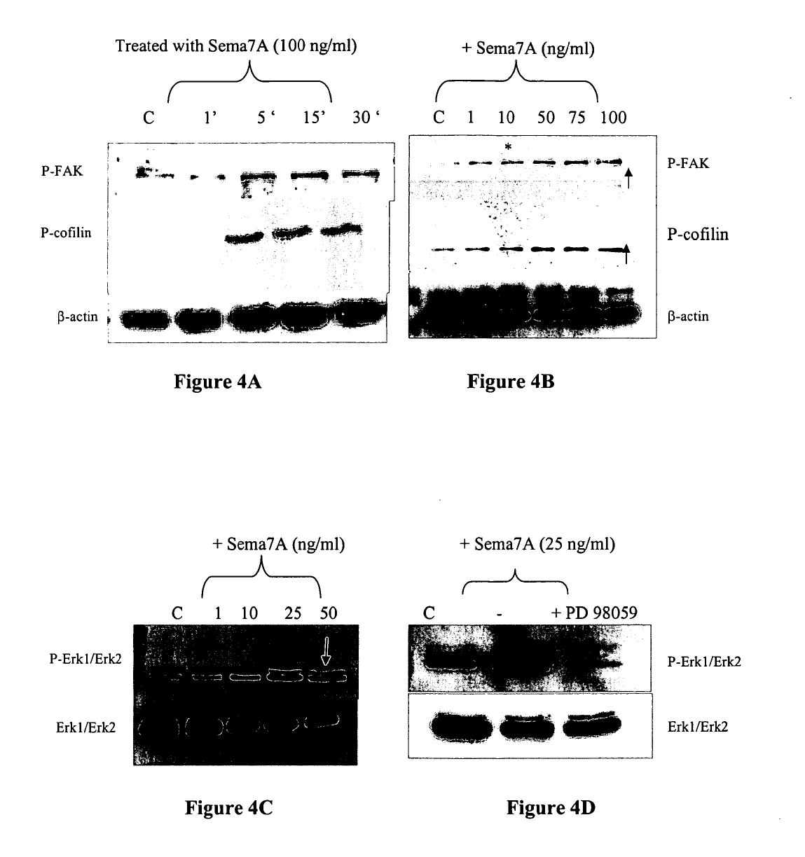

To examine potential mechanisms by which the loss of Plexin C1 expression leads to melanoma progression, the signaling pathways stimulated by Sema7A in normal human melanocytes and the downstream targets of Plexin C1 signaling were analyzed. Cofilin activation is controlled through phosphorylation of the protein at Ser-33, which results in its inactivation (Moriyama et al., 1996). Treatment of melanocytes with Sema7A stimulated the rapid phosphorylation of cofilin, as determined by Western blotting with antibodies against cofilin phosphorylated on Ser-33 (FIG. 4A). Within 5 minutes of treatment with Sema7A, phosphorylation of cofilin occurred, and then began to diminish 30 minutes later.

Previously, it was demonstrated that Sema7A stimulates melanocyte adhesion and dendrite formation through β1-integrin receptors. To determine if Sema7A stimulates integrin signaling, the effect of Sema7A on the activation of the non-receptor protein tyrosine kinase focal adhesion kinase (FAK) was examined. Upon integrin receptor binding, FAK is rapidly phosphorylated, which resulted in propagation of integrin signals (Hehlgans et al., 2007; Richardson and Parsons, 1995). Sema7A stimulated the rapid phosphorylation of FAK in melanocytes, indicative of β1-integrin signaling (FIG. 4A). A dose-response analysis showed that at a dose of 10 ng/ml Sema7A detectable cofilin and FAK phosphorylation occurred. This phosphorylation peaked at a dose of 50 ng/ml (FIG. 4B). Previous studies reported that Sema7A activated MAP kinase in neurons (Huang et al., 2007; Li et al., 2007; Pasterkamp et al., 2003; Yee et al., 2008). To determine if Sema7A activated MAP kinase in melanocytes, cells were treated for 5 minutes with Sema7A (doses ranging from 1 ng/ml to 50 ng/ml). Total cell lysates were then blotted for phosphorylated extracellular regulated kinases (Erk) 1 and 2 (FIG. 4C). Sema7A stimulated the rapid phosphorylation of Erk1/Erk2 at a dose of 10 ng/ml. This phosphorylation peaked at a dose of 25 ng/ml, and was inhibited by the selective p42/44 MAP kinase inhibitor PD 98059 (FIG. 4D).

(4) Plexin C1 Signaling Stimulates Cofillin Phosphorylation/Inactivation in Melanocytes

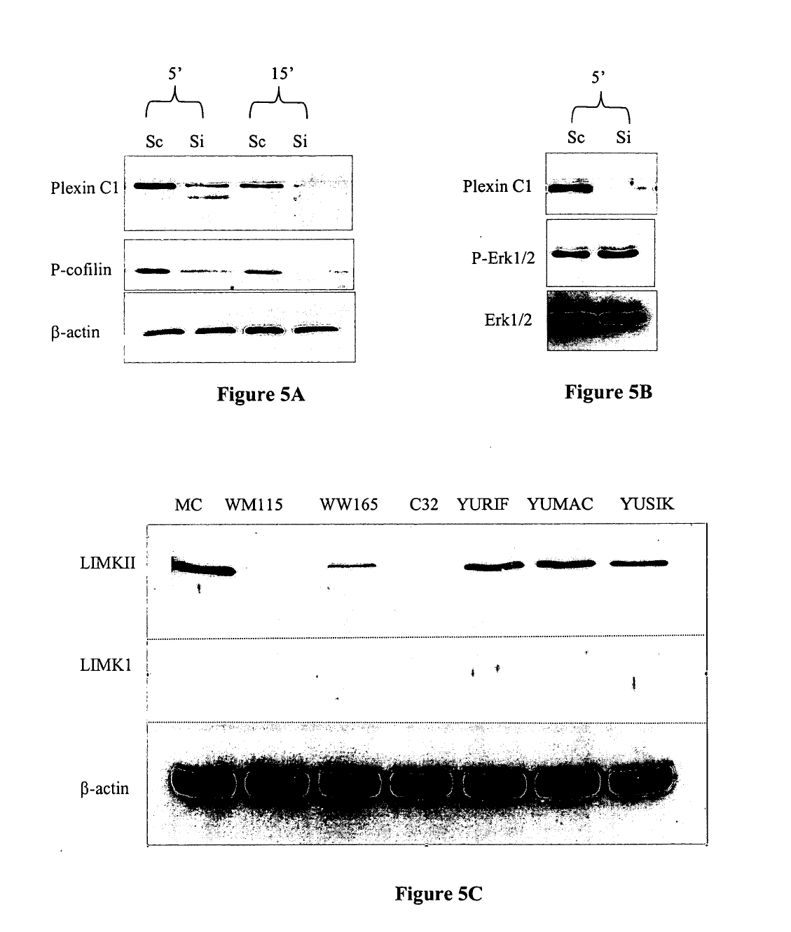

To determine the relationship between Plexin C1 signaling and cofilin phosphorylation, Plexin C1 was silenced in melanocytes using siRNAs. Silenced cells were treated with Sema7A (50 ng/ml) for 5 and 15 minutes. The use of the 5-minute treatment condition produced partial Plexin C1 silencing (>75%). The use the 15 minute treatment condition produced complete silencing (FIG. 5A). The phosphorylation of cofilin in response to Sema7A directly correlated with the levels of Plexin C1 expression. Five minutes post-Sema7A treatment, a time at which residual Plexin C1 expression was still present, the phosphorylation of cofilin was diminished, but not absent. At 15 minutes points-Sema7A treatment, a time at which Plexin C1 expression was fully silenced, the phosphorylation of cofilin was completely abrogated. But, following Sema7A treatment, the silencing of Plexin C1 expression had no effect on Erk1/Erk2 phosphorylation (FIG. 5B).

Cofilin is a direct downstream target of LIM kinases (LIMK), which phosphorylates cofilin on Ser-33. There are 2 isoforms of LIMK, and LIMKI is expressed in neural cells and LIMKII is more widely expressed (Bernard, 2007; Scott and Olson, 2007). To determine which isoform of LIMK is expressed in human melanocytes and in the panel of melanoma cell lines, total cell lysates were blotted for LIMK1 and LIMKII (FIG. 5C). The melanocytes and 3 out of the 5 melanoma cell lines expressed LIMKII, but not LIMKI. Of note, the two cell lines that completely lacked LIMKII expression (WM115 and C32) also lacked Plexin C1. Furthermore, in the melanoma cell lines, the levels of LIMKII expression roughly correlated with the level of Plexin C1 expression (see FIG. 1A).

### c) Discussion

Recent studies showed that the skin expresses Sema7A. There, Sema7A controls the dendricity and adhesion of human melanocytes through the opposing actions of Sema7A-dependent β1-integrin and Plexin C1 signaling (Scott et al., 2008). In that study, the silencing of Plexin C1 enhanced Sema7A′ s effect on melanocyte adhesion and dendricity, which indicated that Plexin C1 signaled to downstream targets that inhibited actin assembly or turnover. Gain or loss of receptor expression, or de novo expression of growth factors, contributes to melanoma initiation and progression (Easty and Bennett, 2000; Haass et al., 2005; Herlyn et al., 1988; Kwong et al., 2007; Meier et al., 2003). Of the 6 human melanoma cell lines examined, Plexin C1 protein expression was completely absent in 2 cell lines, was decreased in 3 cell lines, and was slightly increased in 1 cell line. Levels of Plexin C1 mRNA generally correlated with protein expression; however, the YURIF cell line, which expressed less Plexin C1 protein than did melanocytes, had increased levels of Plexin C1 message. A longer mRNA half-life or attenuated degradation of the protein can produce such a result. Gene silencing that arises from methylation is an important epigenetic mechanism of tumor-suppressor inactivation and can be reversed by the methylation inhibitor 5-Aza2Dc (Kangaspeska et al., 2008; Mellor et al., 2008; Metivier et al., 2008). In response to treatment with 5-Aza2Dc, two melanoma cell lines (YURIF and WW165) showed a dose-dependent decrease in Plexin C1 expression. But, at a 0.1 μM dose of 5-Aza2Dc, one cell line (YUMAC) showed an increase in Plexin C1 expression. These data indicate that, in melanoma cell lines, DNA methylation plays a role in the regulation of Plexin C1 expression. In some cases, such regulation can be indirect (e.g., through silencing of genes that regulate Plexin C1). This means that other mechanisms, such as gene deletion, can be also involved. Apaf-1 is a gene linked to Plexin C1 on chromosome 12, and Apaf-1 expression is also lost during melanoma progression. The loss of heterozygosity contributes to Apaf-1 deletion (Soengas et al., 2001). The complete absence of Plexin C1 message in 2 of the 6 melanoma cell lines indicates that gene deletion can play a role in loss of Plexin C1 expression. To determine if the loss of Plexin C1 expression is due to deletion of part or the entire gene, current studies now examine genomic DNA from metastatic and primary melanomas.

The expression of Plexin C1 by immunohistochemical staining of TMA of benign nevi, melanoma primary to the skin, and metastatic melanoma was examined. Tissue microarrays allowed analysis of multiple samples that are uniformly stained from the same slide (Simon et al., 2004). This data demonstrated a striking loss of Plexin C1 expression in metastatic melanoma, and an inverse correlation between Plexin C1 expression and tumor invasiveness in primary melanomas. In virtually all subjects, nevic cells showed strong homogenous Plexin C1 expression. In contrast, the majority of metastatic melanomas showed weak or no staining for Plexin C1. In primary melanoma, the Plexin C1 expression was intermediate between nevi and metastatic tumors, and Plexin C1 expression was progressively lost in melanomas of increasing depth. Very deep melanomas (>4 mm) showed an average IS of 1.3, compared with thin melanomas (<1 mm) that showed an average IS of 3. Particularly informative was the juxtaposition of a benign nevus and melanoma, in which strong Plexin C1 expression was evident in the nevic cells, but was absent in the melanoma cells (FIG. 3B). In toto, these data support a role for Plexin C1 as a tumor suppressor protein for melanoma progression. Because the level of Plexin C1 expression in benign nevi and superficially invasive melanoma (<1 mm depth of invasion) was essentially the same, the loss of Plexin C1 is likely to promote tumor progression (invasion and metastasis) rather than tumor initiation. Current studies now explore whether the loss of Plexin C1 expression in primary tumors correlates with metastatic disease in individual patients. Thus, Plexin C1 expression is a useful as a prognostic indicator for melanoma metastasis.

Although the relationship between cofilin activation and tumor progression is complex, cofilin activation is strongly linked to tumor progression (Huang et al., 2006; Paavilainen et al., 2004). In metastatic tumor cell lines, the loss of cofilin expression is associated with decreased cell turning and chemotactic sensitivity to epidermal growth factor (Sidani et al., 2007). Furthermore, the cofilin activation pathway is a major determinant of breast cancer metastasis (Wang et al., 2007). While there is abundant data to indicate a role for cofilin activation in carcinogenesis, the data on the role of cofilin and melanoma progression is more limited. The knockdown of cofilin and actin depolymerizing factor in B16F1 melanoma cells inhibited cell migration, which inhibition was primarily accomplished through diminished actin filament depolymerization rates (Hotulainen et al., 2005). In K1735 murine melanoma cells, expression of wild type (non-phosphorylatable) cofilin increased melanoma invasion and migration (Dang et al., 2006). While the mechanisms by which the loss of Plexin C1 promotes melanoma progression are likely multifactorial, these data indicate a potential link between loss of Plexin C1 expression and cofilin activation in melanoma.

At low doses (10 ng/ml), Sema7A stimulated the rapid phosphorylation of cofilin and FAK in human melanocytes. Phosphorylation of FAK, which occurs upon integrin activation, is consistent with prior data showing that Sema7A dependent melanocyte adhesion occurs through the engagement of β1-integrins (Scott et al., 2008). Sema7A also activated MAP kinase in normal human melanocytes, and was unaffected by Plexin C1 silencing. As reported in neural cells, bone cells and monocytes, it is likely that MAP kinase activation is secondary to Sema7A-dependent integrin activation (Delorme et al., 2005; Pasterkamp et al., 2003; Suzuki et al., 2007). Current experiments explore this likelihood. To address whether Plexin C1 regulates cofilin phosphorylation, Plexin C1 was silenced in human melanocytes, and the effect of Sema7A on cofilin phosphorylation was examined. When Plexin C1 was silenced, Sema7A dependent cofilin phosphorylation was lost or attenuated, which indicated that Plexin C1 regulated cofilin phosphorylation. The LIMK family of proteins consists of two members, LIMKI, and LIMKII, which phosphorylate cofilin at Ser-33 (Bernard 2007; Scott and Olson, 2007). LIMKI is primarily expressed in the central nervous system whereas LIMKII is widely expressed (Scott and Olson, 2007). Normal melanocytes, and four of the six melanoma cell lines examined, expressed LIMKII, but not LIMKI. Hence, in melanocytic cells, LIMKII is likely a downstream target of Plexin C1 signaling. The examination of cofilin regulation in melanocytes expressing kinase dead mutants of LIMKII is required to address the role of LIMKII in Plexin C1-dependent cofilin regulation. Of interest was the precise correlation between levels LIMKII and Plexin C1 expression: melanoma cell lines lacking Plexin C1 (C32 and WM115) also lacked LIMKII. Similarly, the expression levels of LIMKII protein closely paralleled the expression levels of Plexin C1. Because LIMKII and Plexin C1 genes are located on different chromosomes (22q12 and 12q23 respectively), a coordinated loss of Plexin C1 and LIMKII is unlikely. It is possible that Plexin C1 and LIMKII share transcriptional regulatory sites that coordinate their expression.

In summary, these studies demonstrated that Plexin C1, a receptor for Sema7A, was decreased or lost during melanoma progression in vivo, and that Plexin C1 signaling in normal human melanocytes resulted in phosphorylation and inactivation of cofilin. DNA hypermethylation was a regulatory mechanism for Plexin C1 expression. Also, Sema7A activated MAP kinase. These data are the first to define the signaling intermediates of Sema7A in human melanocytes, and point to a role for Plexin C1 as a novel tumor suppressor protein for melanoma progression, potentially through loss of inhibition of cofilin activation.

d) Material and Methods

(1) Reagents:

Rabbit polyclonal antibodies to β-actin, goat polyclonal antibodies to Plexin C1, and rabbit polyclonal antibodies to LIMKI and LIMKII were purchased from Santa Cruz Biotechnology (Santa Cruz, Calif.); rabbit polyclonal antibodies to cofilin phosphorylated on Ser-33, rabbit polyclonal antibodies to phospho-p44/42 MAP kinase, and mouse monoclonal antibodies to p42 MAP kinase were purchased from Cell Signaling Technology (Danvers, Mass.), mouse monoclonal antibodies to FAK phosphorylated on Y397 were purchased from Chemicon International (Temecula, Calif.). Horseradish peroxidase-conjugated goat anti-rabbit and goat anti-mouse antibodies were purchased from Sigma Co (St. Louis, Mo.). FITC-conjugated mouse anti-human Sema7A (CDw108) and mouse IgM conjugated to FITC for flow cytometry were purchased from Serotec (Kidlington, Oxford, UK). Full range rainbow molecular weight markers were purchased from Amersham Life Sciences (Arlington Heights, Ill.). Silencing RNAs (siRNA) to human Plexin C1, Silencer Negative Control no. 1 siRNA, and lipofectamine were purchased from Ambion (Austin, Tex.). Sema7A, in frame with human Fc fragment, in pcDNA3 vector was a generous gift from Dr Ruslan Medzhitov (Section of Immunobiology, Yale University, New Haven Conn.) and has been described previously (Czopik et al., 2006). The DNA methylation inhibitor 5-Aza-2′-Deoxycytidine (5-Aza2Dc) and selective p42/44 MAP kinase inhibitor PD 98059 were purchased from Sigma Co.

(2) Cells and Cell Culture:

Neonatal foreskins were obtained according to the University of Rochester Research Subjects Review Board guidelines and were the source of cultured human melanocytes. Human melanocytes were cultured in MCDB 153 supplemented with 0.5% fetal bovine serum (FBS), bovine pituitary extract (15 μg/ml), phorbol ester (10 nM), basic fibroblast growth factor (1 ng/ml), insulin (5 μg/ml) and hydrocortisone (500 ng/ml). All supplements were purchased from Sigma Co, except FBS, which was purchased from Mediatech (Manassas, Va.). The following human melanoma cell lines were purchased from the Yale University Cell Core Facility: WW165, YURIF, YUSIK, YUMAC, and were maintained in Opti-MEM+5% FBS, except WW165 which was maintained in Opti-MEM+5% FBS and IBMX 0.1 mM. The C32 and WM115 human melanoma cell lines were obtained from American Type Culture Collection (Manassas Va.) and were maintained in Eagle Minimal Essential Media (MEM)+10% FBS and 1 mM sodium pyruvate. The WM115 and WW 165 cell lines were derived from melanomas primary to the skin; the other cell lines were derived from metastatic melanoma.

(3) Purification of Fc-Tagged Sema7A:

Fc-tagged Sema7A was isolated from culture supernatant of stable transfectants of COS-1 cells expressing Fc-Sema7A, on HiTrap protein A HP columns (General Electric Healthcare, St. Giles, UK) as per manufacturer's instructions. Eluent was resolved on a 7.5% SDS PAGE and blotted for Sema7A to verify its identity as previously described (Scott et al., 2008). Coomassie-stained gels showed a single band corresponding to the molecular weight of Sema7A at a protein concentration of 1 mg/ml.

(4) Construction, Staining and Analysis of Tissue Microarrays:

The project received IRB exemption from the Human Subjects Review Board at the University of Rochester: Category 4 (45 CRF 46.101): secondary use of pre-existing data. Twenty-six cases of benign nevi, 27 cases of melanoma primary to the skin, and 24 cases of metastatic melanoma were chosen from formalin fixed, paraffin embedded archival material from Strong Memorial Department of Pathology. One of the authors (GS) diagnosed each case during clinical duties as Director of Dermatopathology at Strong Memorial Hospital, Rochester N.Y. The slides from each case were re-examined and 3 representative areas from each slide were chosen. Three 1 mm cores corresponding to these areas were removed from the paraffin block and placed in a recipient block. All benign nevi were dermal nevi with a minimal junctional component, primary melanomas had depths of <1 mm (n=9), between 1.01 and 2.0 mm (n=8), between 2.01 and 4.0 mm (n=6) and >4 mm (n=4). For metastatic melanoma, 11/24 cases were metastatic to lymph nodes, 5/24 were metastatic to skin and 8/24 cases were metastatic to organs including gastrointestinal system, lung and bone.

(5) Staining of TMA for Plexin C1:

Sections were de-paraffinized through graded series of alcohol, rehydrated in water, and endogenous peroxidase was quenched by incubation in 3% H₂O₂ for 3 min×2. Non-specific staining was blocked by incubation in non-protein blocking solution (Dako, Carpenteria, Calif.) for 15 min at room temperature. Antigen retrieval was performed by incubating the slides in Target retrieval Buffer (Dako) at 98° C. for 15 minutes. Slides were incubated with polyclonal antibodies against Plexin C1 in antibody diluent (1/100, Dako) for 1 hour at room temperature. Sections were then incubated with biotin-conjugated rabbit anti-goat antibodies for 30 minutes at room temperature followed by streptavidin horseradish-peroxidase (Vector Laboratories, Burlingame, Calif.) for 20 minutes. The reaction was developed with amino-ethyl carbizol and the slides were counterstained in Mayer's hematoxylin. Negative controls consisted of goat IgG (Sigma Co.) instead of the primary antibody.

(6) Analysis of TMAs:

The intensity of staining of each 1 mm core was rated on a scale of 0 to 3, with 0=negative; 1=weak; 2=moderate, and 3=strong. For all cases, when staining was present, it was diffuse, with all nevi cells or melanoma cells expressing uniform staining for Plexin C1. Each core was examined under a light microscope by one of the authors (GS) and separately scored, and the staining intensity of the cores averaged to arrive at an intensity score (IS). Cores with <50% of designated tissue present were disregarded. Cases with an average IS score of 0 to 1.5 were considered negative; those with an IS score between 1.6 and 2.5 were considered moderately positive, and those with an IS score of 2.6 to 3 were considered strongly positive.

(7) Statistical Evaluation of Data:

Data were summarized in terms of their mean and standard deviation. Tissue expression of Plexin C1 was analyzed using Fisher's exact test to determine statistically significant difference in expression between nevi, primary melanoma, metastatic melanoma, and in melanomas of different Breslow depths (0.1-1 mm, 1.01-2.0 mm, 2.1-4 mm and >4.0 mm). A p value of <0.05 was considered significant.

(8) Western Blotting and Flow Cytometry:

For Western blotting, cells were lysed in RIPA buffer (150 mM NaCl, 1% NP-40, 0.5% DOC, 0.1% SDS, 50 mM Tris-HCl) with protease inhibitors (Boehringer Mannheim, Gmbt, Germany) and phosphatase inhibitors (Gbiosciences, St. Louis, Mo.) and protein was quantified using bovine serum albumen (BSA) as standard (Bio-Rad Laboratories, Hercules, Calif.). Visualization of the immunoreactive proteins was accomplished with an enhanced chemiluminescence reaction (Pierce Chemical, Rockford, Ill.). For analysis of cell surface Sema7A expression in melanoma cell lines by flow cytometry, cells were placed in suspension in PBS/1% BSA and stained with FITC-conjugated antibodies against Sema7A for 30 minutes at room temperature. Negative controls consisted of cells incubated with FITC-conjugated IgM. After washing in PBS, cells were re-suspended in 0.5% formalin in PBS/1% BSA and analyzed using a FACS Caliber Machine (Becton Dickenson) at the flow cytometry core facility at the University of Rochester.

(9) Quantitative Real Time PCR:

Total RNA was isolated using the RNeasy Mini Kit (QIAgen, Valencia, Calif.) according to manufacturer's instructions. Reverse transcription was performed using 0.75 μg of total RNA with SuperScript II reverse transcriptase (Invitrogen, Carlsbad, Calif.). PCR was performed using iQ SYBR Green Supermix (BioRad Laboratories) on the Applied Biosystems ABI prism 7700 sequence-detection system (BioRad iCycler). Primers used for amplification of Plexin C1 were: fwd: 5′-AACCATTGCACTGCAACC-3′ (SEQ ID NO: 5); rvs: 5′-GATTCCATCTTCAAGAATCACG-3′(SEQ ID NO: 6). The conditions were: 95° C., 3 min (1 cycle); 95° C. 15 sec, 54.5° C., 30 sec, 72° C., 40 sec (40 cycles). Primers used for amplification of β-actin were: fwd: 5′-CACGCACGATTTCCCGCTCGG-3′(SEQ ID NO: 7); rvs: 5′-CAGGCTGTGCTATCCTGTAC-3′(SEQ ID NO: 8). The conditions were 95° C., 3 min (1 cycle); 95° C. 15 sec, 54.5° C., 30 sec, 72° C., 40 sec (40 cycles). The PCR product was resolved on 1% agarose gels, sequenced and verified. Quantification of PCR product was determined by analyzing a standard curve of known amounts of Plexin C1 PCR product to unknown samples. Samples were then normalized to β-actin.

### E. REFERENCES

Arnaout M A, Goodman S L, Xiong J P (2007) Structure and mechanics of integrin-based cell adhesion. Current opinion in cell biology 19:495-507.

Artigiani S, Comoglio P M, Tamagnone L (1999) Plexins, semaphorins, and scatter factor receptors: a common root for cell guidance signals? IUBMB life 48:477-482.

Balch C M, Wilkerson J A, Murad T M, Soong S J, Ingalls A L, Maddox W A (1980) The prognostic significance of ulceration of cutaneous melanoma. Cancer 45:3012-3017.

Basile J R, Barac A, Zhu T, Guan K L, Gutkind J S (2004) Class IV semaphorins promote angiogenesis by stimulating Rho-initiated pathways through plexin-B. Cancer Res 64:5212-5224.

Basile J R, Castilho R M, Williams V P, Gutkind J S (2006) Semaphorin 4D provides a link between axon guidance processes and tumor-induced angiogenesis. Proceedings of the National Academy of Sciences of the United States of America 103:9017-9022.

Bernard 0 (2007) Lim kinases, regulators of actin dynamics. The international journal of biochemistry & cell biology 39:1071-1076.

Bielenberg DR, Hida Y, Shimizu A, Kaipainen A, Kreuter M, Kim C C, et al. (2004) Semaphorin 3F, a chemorepulsant for endothelial cells, induces a poorly vascularized, encapsulated, nonmetastatic tumor phenotype. The Journal of clinical investigation 114:1260-1271.

Bielenberg DR, Klagsbrun M (2007) Targeting endothelial and tumor cells with semaphorins. Cancer metastasis reviews 26:421-431.

Czopik A K, Bynoe M S, Palm N, Raine C S, Medzhitov R (2006) Semaphorin 7A is a negative regulator of T cell responses. Immunity 24:591-600.

Dahl C, Guldberg P (2007) The genome and epigenome of malignant melanoma. Apmis 115:1161-1176.

Dang D, Bamburg J R, Ramos DM (2006) Alphavbeta3 integrin and cofilin modulate K1735 melanoma cell invasion. Experimental cell research 312:468-477.

Delorme G, Saltel F, Bonnelye E, Jurdic P, Machuca-Gayet I (2005) Expression and function of semaphorin 7A in bone cells. Biol Cell 97:589-597.

Easty D J, Bennett D C (2000) Protein tyrosine kinases in malignant melanoma. Melanoma research 10:401-411.

Giger R J, Pasterkamp R J, Holtmaat A J, Verhaagen J (1998) Semaphorin III: role in neuronal development and structural plasticity. Prog Brain Res 117:133-149.

Gronbaek K, Hother C, Jones P A (2007) Epigenetic changes in cancer. Apmis 115:1039-1059.

Haass N K, Smalley K S, Li L, Herlyn M (2005) Adhesion, migration and communication in melanocytes and melanoma. Pigment cell research/sponsored by the European Society for Pigment Cell Research and the International Pigment Cell Society 18:150-159.

Hamoen K E, Borel Rinkes I H, Morgan JR (2001) Hepatocyte growth factor and melanoma: gene transfer studies in human melanocytes. Melanoma research 11:89-97.

Hehlgans S, Haase M, Cordes N (2007) Signalling via integrins: implications for cell survival and anticancer strategies. Biochimica et biophysica acta 1775:163-180.

Herlyn M, Mancianti M L, Jambrosic J, Bolen J B, Koprowski H (1988) Regulatory factors that determine growth and phenotype of normal human melanocytes. Experimental cell research 179:322-331.

Hess A R, Postovit L M, Margaryan N V, Seftor E A, Schneider G B, Seftor R E, et al. (2005) Focal adhesion kinase promotes the aggressive melanoma phenotype. Cancer research 65:9851-9860.

Hotulainen P, Paunola E, Vartiainen M K, Lappalainen P (2005) Actin-depolymerizing factor and cofilin-1 play overlapping roles in promoting rapid F-actin depolymerization in mammalian nonmuscle cells. Molecular biology of the cell 16:649-664.

Hu B, Guo P, Bar-Joseph I, Imanishi Y, Jarzynka M J, Bogler O, et al. (2007) Neuropilin-1 promotes human glioma progression through potentiating the activity of the HGF/SF autocrine pathway. Oncogene 26:5577-5586.

Huang D, Khoe M, Befekadu M, Chung S, Takata Y, Ilic D, et al. (2007) Focal adhesion kinase mediates cell survival via NF-kappaB and ERK signaling pathways. American journal of physiology 292:C1339-1352.

Huang T Y, DerMardirossian C, Bokoch G M (2006) Cofilin phosphatases and regulation of actin dynamics. Current opinion in cell biology 18:26-31.

Huveneers S, Truong H, Danen H J (2007) Integrins: signaling, disease, and therapy. International journal of radiation biology 83:743-751.

Ishida I, Kumanogoh A, Suzuki K, Akahani S, Noda K, Kikutani H (2003) Involvement of CD100, a lymphocyte semaphorin, in the activation of the human immune system via CD72: implications for the regulation of immune and inflammatory responses. Int Immunol 15:1027-1034.

Kangaspeska S, Stride B, Metivier R, Polycarpou-Schwarz M, Ibberson D, Carmouche R P, et al. (2008) Transient cyclical methylation of promoter DNA. Nature 452:112-115.

Kessler O, Shraga-Heled N, Lange T, Gutmann-Raviv N, Sabo E, Baruch L, et al. (2004) Semaphorin-3F is an inhibitor of tumor angiogenesis. Cancer Res 64:1008-1015.

Klein C E, Steinmayer T, Kaufmann D, Weber L, Brocker E B (1991) Identification of a melanoma progression antigen as integrin VLA-2. The Journal of investigative dermatology 96:281-284.

Koh J M, Oh B, Lee J Y, Lee J K, Kimm K, Kim G S, et al. (2006) Association study of semaphorin 7a (sema7a) polymorphisms with bone mineral density and fracture risk in postmenopausal Korean women. J Hum Genet. 51:112-117.

Kreuter M, Bielenberg D, Hida Y, Hida K, Klagsbrun M (2002) Role of neuropilins and semaphorins in angiogenesis and cancer. Annals of hematology 81 Suppl 2:S74.

Kwong L, Chin L, Wagner S N (2007) Growth factors and oncogenes as targets in melanoma: lost in translation? Advances in dermatology 23:99-129.

Li Y, Gallant C, Malek S, Morgan K G (2007) Focal adhesion signaling is required for myometrial ERK activation and contractile phenotype switch before labor. Journal of cellular biochemistry 100:129-140.

Matsumoto K, Ziober B L, Yao CC, Kramer R H (1995) Growth factor regulation of integrin-mediated cell motility. Cancer Metastasis Rev 14:205-217.

Maurin JC, Delorme G, Machuca-Gayet I, Couble M L, Magloire H, Jurdic P, et al. (2005) Odontoblast expression of semaphorin 7A during innervation of human dentin. Matrix Biol 24:232-238.

Meier F, Caroli U, Satyamoorthy K, Schittek B, Bauer J, Berking C, et al. (2003) Fibroblast growth factor-2 but not MeI-CAM and/or beta3 integrin promotes progression of melanocytes to melanoma. Exp Dermatol 12:296-306.

Mellor J, Dudek P, Clynes D (2008) A glimpse into the epigenetic landscape of gene regulation. Current opinion in genetics & development.

Metivier R, Gallais R, Tiffoche C, Le Peron C, Jurkowska R Z, Carmouche R P, et al. (2008) Cyclical DNA methylation of a transcriptionally active promoter. Nature 452:45-50.

Moretti S, Procopio A, Boemi M, Catalano A (2006) Neuronal semaphorins regulate a primary immune response. Curr Neurovasc Res 3:295-305.

Moriyama K, Iida K, Yahara I (1996) Phosphorylation of Ser-3 of cofilin regulates its essential function on actin. Genes Cells 1:73-86.

Paavilainen V O, Bertling E, Falck S, Lappalainen P (2004) Regulation of cytoskeletal dynamics by actin-monomer-binding proteins. Trends Cell Biol 14:386-394.

Pasterkamp R J (2005) R-Ras fills another GAP in semaphorin signalling. Trends Cell Biol 15:61-64.

Pasterkamp R J, De Winter F, Holtmaat A J, Verhaagen J (1998a) Evidence for a role of the chemorepellent semaphorin III and its receptor neuropilin-1 in the regeneration of primary olfactory axons. J Neurosci 18:9962-9976.

Pasterkamp R J, Giger R J, Verhaagen J (1998b) Regulation of semaphorin III/collapsin-1 gene expression during peripheral nerve regeneration. Exp Neurol 153:313-327.

Pasterkamp R J, Peschon J J, Spriggs M K, Kolodkin A L (2003) Semaphorin 7A promotes axon outgrowth through integrins and MAPKs. Nature 424:398-405.

Pasterkamp R J, Ruitenberg M J, Verhaagen J (1999) Semaphorins and their receptors in olfactory axon guidance. Cell Mol Biol (Noisy-1e-grand) 45:763-779.

Pasterkamp R J, Verhaagen J (2006) Semaphorins in axon regeneration: developmental guidance molecules gone wrong? Philos Trans R Soc Lond B Biol Sci 361:1499-1511.

Patrick R J, Corey S, Glass L F (2007) The use of sequential serial sectioning of thin melanomas in determining maximum Breslow depth. Journal of the American Academy of Dermatology 57:S127-128.

Richardson A, Parsons J T (1995) Signal transduction through integrins: a central role for focal adhesion kinase? Bioessays 17:229-236.

Rothhammer T, Bosserhoff A K (2007) Epigenetic events in malignant melanoma. Pigment cell research/sponsored by the European Society for Pigment Cell Research and the International Pigment Cell Society 20:92-111.

Scott G A, McClelland L A, Fricke A F (2008) Semaphorin 7a promotes spreading and dendricity in human melanocytes through beta1-integrins. The Journal of investigative dermatology 128:151-161.

Scott R W, Olson M F (2007) LIM kinases: function, regulation and association with human disease. J Mol Med 85:555-568.

Sidani M, Wessels D, Mouneimne G, Ghosh M, Goswami S, Sarmiento C, et al. (2007) Cofilin determines the migration behavior and turning frequency of metastatic cancer cells. The Journal of cell biology 179:777-791.

Simon R, Mirlacher M, Sauter G (2004) Tissue microarrays. BioTechniques 36:98-105.

Soengas M S, Capodieci P, Polsky D, Mora J, Esteller M, Opitz-Araya X, et al. (2001) Inactivation of the apoptosis effector Apaf-1 in malignant melanoma. Nature 409:207-211.

Suzuki K, Okuno T, Yamamoto M, Pasterkamp R J, Takegahara N, Takamatsu H, et al. (2007) Semaphorin 7A initiates T-cell-mediated inflammatory responses through alpha1beta1 integrin. Nature 446:680-684.

Swiercz J M, Kuner R, Offermanns S (2004) Plexin-B1/RhoGEF-mediated RhoA activation involves the receptor tyrosine kinase ErbB-2. The Journal of cell biology 165:869-880.

Takagi J (2007) Structural basis for ligand recognition by integrins. Current opinion in cell biology 19:557-564.

Tamagnone L, Artigiani S, Chen H, He Z, Ming G I, Song H, et al. (1999) Plexins are a large family of receptors for transmembrane, secreted, and GPI-anchored semaphorins in vertebrates. Cell 99:71-80.

Tamagnone L, Comoglio P M (2000) Signalling by semaphorin receptors: cell guidance and beyond. Trends Cell Biol 10:377-383.

Walzer T, Galibert L, Comeau MR, De Smedt T (2005a) Plexin C1 engagement on mouse dendritic cells by viral semaphorin A39R induces actin cytoskeleton rearrangement and inhibits integrin-mediated adhesion and chemokine-induced migration. J Immunol 174:51-59.

Walzer T, Galibert L, De Smedt T (2005b) Dendritic cell function in mice lacking Plexin C1. Int Immunol 17:943-950.

Wang W, Eddy R, Condeelis J (2007) The cofilin pathway in breast cancer invasion and metastasis. Nature reviews 7:429-440.

Yamada T, Endo R, Gotoh M, Hirohashi S (1997) Identification of semaphorin E as a non-MDR drug resistance gene of human cancers. Proceedings of the National Academy of Sciences of the United States of America 94:14713-14718.

Yee K L, Weaver V M, Hammer DA (2008) Integrin-mediated signalling through the MAP-kinase pathway. IET systems biology 2:8.

What is claimed is:

1. A method of determining the prognosis of a patient with cancer comprising obtaining a tissue sample from the subject and measuring the level of Plexin C1 in the sample, wherein a decrease in the level of Plexin C1 relative to a normal control indicates a more aggressive cancer.

2. The method of claim 1, wherein the level of Plexin C1 is determined by measuring Plexin C1 protein or mRNA expression.

3. The method of claim 2, wherein Plexin C1 protein expression is measured by Western blotting, flow cytometry, Immunohistochemical staining, ELISA, ELISPOT, or real time PCR analysis.

4. The method of claim 2, wherein the level of Plexin C1 mRNA expression is measured by Quantitative PCT or microarray.

5. A method of characterizing the metastatic potential of a cancer comprising obtaining a tissue sample from the subject and measuring the level of Plexin C1 in the sample, wherein a decrease in the level of Plexin C1 relative to a normal control indicates a cancer with a greater metastatic potential.

6. A method of assessing the aggressiveness of a cancer comprising obtaining a tissue sample from the subject and measuring the level of Plexin C1 in the sample, wherein a decrease in the level of Plexin C1 relative to a normal control indicates a more aggressive cancer.

7. A method of assessing cancer progression comprising obtaining a tissue sample from a subject and measuring the level of Plexin C1 in the sample, wherein a decrease in the level of Plexin C1 relative to a normal control indicates an increased progression of the cancer.

8. A method of determining the aggressiveness of a treatment of a cancer comprising obtaining a tissue sample from the subject and measuring the level of Plexin C1 in the sample, wherein a decrease in the level of Plexin C1 relative to a normal control indicates a more aggressive treatment should be used to treat the cancer.

9. A kit for measuring Plexin C1 levels in a cancer in a subject comprising a first agent that specifically binds to Plexin C1, one or more reagents for detecting the presence of the first agent, and a control.

10. The kit of claim 9, wherein the agent that binds Plexin C1 is an antibody.

11. The kit of claim 10, wherein the antibody to Plexin C1 has a detectable label.

12. The kit of claim 11, wherein the detectable label is biotin.

13. The kit of claim 9, further comprising a second agent which binds to the first agent.

14. A method of inhibiting a metastatic progression of cancer in a subject comprising administering to the subject an agent that inhibits actin assembly.

15. The method of claim 14, wherein the agent that inhibits actin assembly is Plexin C1.

16. The method of claim 14, wherein the Plexin C1 is administered in a viral vector.

17. A method of inhibiting a metastatic progression of cancer in a subject comprising administering to the subject an agent that increases Plexin C1 expression or signaling.

18. The method of claim 17, wherein the agent that increases Plexin C1 expression or signaling is exogenous Plexin C1.

19. The method of claim 18, wherein the exongenous Plexin C1 is administered in a viral vector comprising Plexin C1.

20. The method of claim 17, wherein the agent that increases Plexin C1 signaling is Sema7a.

21. The method of claim 20, wherein the amount of Sema7a administered is between 10 ng/mL and 100 ng/mL.

22. The method of claim 21, wherein the amount of Sema7a administered is 50 ng/mL.

23. A method of inhibiting melanoma progression in a subject comprising administering to the subject an agent that inhibits actin assembly.

24. A method of treating a cancer comprising administering to the subject an agent that inhibits actin assembly.

25. A method of inhibiting cofilin activation comprising administering to the subject an agent that phosphorylates cofilin.

26. The method of claim 25, wherein the agent is Plexin C1.
